(19) United States
(12) Patent Application Publication
(10) Pub. No.: US 2008/0086056 A1
(43) Pub. Date: Apr. 10, 2008

(54) MICRO ULTRASONIC TRANSDUCERS

(75) Inventors: Ming-Wei CHANG, Taichung Hsien (TW); Hsu-Cheng DENG, Hsinchu City (TW); Tien-Chu FAN, Taichung City (TW); Chih-Min LAI, Miaoli County (TW); Gwo-Shiang LEE, Taipei Hsien (TW); Chia-Lin CHIU, Taipei Hsien (TW)

Correspondence Address: Akin Gump LLP - Silicon Valley, 3000 El Camino Real, Two Palo Alto Square, Suite 400, Palo Alto, CA, 94306, US

(73) Assignee: INDUSTRIAL TECHNOLOGY RESEARCH INSTITUTE, Hsinchu (TW)

(21) Appl. No.: 11/870,396

(22) Filed: Oct. 10, 2007

Related U.S. Application Data
(63) Continuation-in-part of application No. 10/648,495, filed on Aug. 25, 2003, now abandoned.

(51) Int. Cl. A61B 8/13

(52) U.S. Cl. 600/459

(57) ABSTRACT

An ultrasonic transducer comprising a flexible membrane, a first electrode in the flexible membrane, a second electrode in the flexible membrane, and a chamber in the flexible membrane between the first electrode and the second electrode.

### CROSS-REFERENCE TO RELATED APPLICATIONS

This application is a continuation-in-part of U.S. patent application Ser. No. 10/648,495, filed Aug. 25, 2003, which is herein incorporated by reference.

### FIELD OF THE INVENTION

The present invention relates generally to an ultrasonic transducer and, more particularly, to a flexible capacitive ultrasonic transducer.

### BACKGROUND OF THE INVENTION

Ultrasonic sensing devices have been widely used in medical, military and aerospace industries because they have the advantages of non-invasive evaluation, real-time response, relatively high resolution and portability. For example, ultrasonic imaging systems are capable of obtaining information from surrounding means or from human body, based on the use of elastic waves at ultrasonic frequency. An ultrasonic transducer is often one of the important components in an ultrasonic sensing device. The majority of known ultrasonic transducers are realized by using piezoelectric ceramic. A piezoelectric transducer is generally used to obtain information from solid materials because the acoustic impedance of piezoelectric ceramic is of the same magnitude order as those of the solid materials. However, the piezoelectric transducer may not be ideal for obtaining information from fluids because of significant impedance mismatch between piezoelectric ceramic and fluids, for example, tissues of the human body.

FIG. 1 is a schematic diagram illustrating the issue of attenuation with an ultrasonic transducer 10 in the prior art. Referring to FIG. 1, the ultrasonic transducer 10 may transmit an ultrasonic wave 12 through a medium toward an object 14 having a curved surface 16, and receive a reflected wave 18 from the curved surface 16 of the object 14. The medium may include a solid or fluid, which may attenuate the ultrasonic wave 12 and the reflected wave 18. Furthermore, the effective sensor area, where a reflected wave is able to be received by the ultrasonic transducer 10, may depend on the surface topology of the object 14. In the present example, as compared to a planar surface, the effective sensor area of the curved surface 16, for example, the skin of a human body, may be reduced. Generally, the attenuation may become more significant and the effective sensor area may decrease as the distance between the ultrasonic transducer 10 and the object 14 increases.

FIG. 2A is a schematic diagram illustrating the issue of impedance mismatch with an ultrasonic transducer 20 in the prior art. Referring to FIG. 2A, the ultrasonic transducer 20 may contact an object 24 having a curved surface 24-1 but may not be able to conform to the curved surface 24-1 of the object 24, which may incur the issue of impedance mismatch. For the purpose of illustration, it may be assumed that an impedance level at a contact region 24-2 where the ultrasonic transducer 20 contacts the curved surface 24-1 is Z_{UT}, which may be approximately an inherent impedance of the ultrasonic transducer 20. At a non-contact region 24-3, however, an additional medium such as air or other couplant may exist. Such an additional medium may cause an extra impedance Z_{M}, resulting in impedance mismatch between the contact region 24-2 and the non-contact region 24-3.

FIG. 2B is a diagram illustrating refraction of an ultrasonic wave in accordance with Snell's law. Referring to FIG. 2B, refraction may occur when an incident ultrasonic wave 22 passes through an interface 25 between two media of different refractive indices. For example, the incident ultrasonic wave 22 may travel in air while a refractive ultrasonic wave 23 may travel in tissue. The angle of incidence “θ” may be smaller than the angle of refraction “θ₁” when the refractive index of the tissue is smaller than that of air. The refraction may cause attenuation of the refractive ultrasonic wave 23 as compared to the incident ultrasonic wave 22 in amplitude. The refraction and in turn the attenuation may be aggravated when the interface 25 is a curved surface.

### BRIEF SUMMARY OF THE INVENTION

A novel ultrasonic transducer is disclosed, which may obviate one or more problems resulting from the limitations and disadvantages of the prior art.

Examples of the present invention may provide an ultrasonic transducer comprising a flexible membrane, a first electrode in the flexible membrane, a second electrode in the flexible membrane, and a chamber in the flexible membrane between the first electrode and the second electrode.

Some examples of the present invention may also provide an ultrasonic transducer comprising a first flexible layer, a first conductive layer over the first flexible layer, a second flexible layer over the first conductive layer, a chamber in the second flexible layer, a third flexible layer over the chamber, and a second conductive layer in the third flexible layer. A portion of the third flexible layer between the second conductive layer and the chamber is capable of generating acoustic waves in response to a voltage applied across the first conductive layer and the second conductive layer.

Examples of the present invention may further provide an ultrasonic transducer array comprising a flexible substrate, a first conductive layer on the flexible substrate, and a number of ultrasonic transducers on the first conductive layer. Each of the number of ultrasonic transducers comprises a flexible membrane over the first conductive layer, a second conductive layer in the flexible membrane, and a chamber in the membrane between the first conductive layer and the second conductive layer.

Examples of the present invention may still provide an ultrasonic transducer array comprising a first flexible layer, a first conductive layer on the first flexible layer, and a number of ultrasonic transducers on the first conductive layer. Each of the number of ultrasonic transducers comprises a second flexible layer over the first conductive layer, a chamber in the second flexible layer, a third flexible layer over the chamber, and a second conductive layer in the third flexible layer. A portion of the third flexible layer between the second conductive layer and the chamber is capable of generating acoustic waves in response to a voltage applied across the first conductive layer and the second conductive layer.

Additional features and advantages of the present invention will be set forth in part in the description which follows, and in part will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one example of the present invention and together with the description, serves to explain the principles of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings examples which are presently preferred. It should be understood, however, that the invention is not limited to the precise arrangements and instrumentalities shown.

In the drawings:

FIG. 1 is a schematic diagram illustrating the issue of attenuation with an ultrasonic transducer in the prior art;

FIG. 2A is a schematic diagram illustrating the issue of impedance mismatch with an ultrasonic transducer in the prior art;

FIG. 2B is a diagram illustrating refraction of an ultrasonic wave in accordance with Snell's law;

FIGS. 3A to 3F are cross-sectional diagrams illustrating a method of manufacturing an ultrasonic transducer consistent with an example of the present invention;

FIG. 3A-1 is a cross-sectional diagram illustrating a method of manufacturing an ultrasonic transducer consistent with another example of the present invention;

FIG. 4A is a cross-section view of an ultrasonic transducer consistent with an example of the present invention;

FIG. 4B is a cross-section view of an ultrasonic transducer consistent with another example of the present invention;

FIG. 5 is a three-dimensional view of an ultrasonic transducer array consistent with an example of the present invention;

FIG. 6A is a schematic diagram illustrating the operation of an ultrasonic transducer consistent with an example of the present invention;

FIG. 6B is a diagram illustrating ultrasonic waves from an array of the ultrasonic transducers illustrated in FIG. 6A;

FIG. 7 is a perspective view of an ultrasonic transducer consistent with an example of the present invention; and

FIG. 8 is a top planar view of an ultrasonic transducer array consistent with an example of the present invention.

### DETAILED DESCRIPTION OF THE INVENTION

In this detailed description, for purposes of explanation, numerous specific details are set forth to illustrate examples of the present invention. One skilled in the art will appreciate, however, that examples of the present invention may be practiced without these specific details. Furthermore, one skilled in the art can readily appreciate that the specific sequences in which methods are presented and performed are illustrative and it is contemplated that the sequences can be varied and still remain within the spirit and scope of embodiments of the present invention.

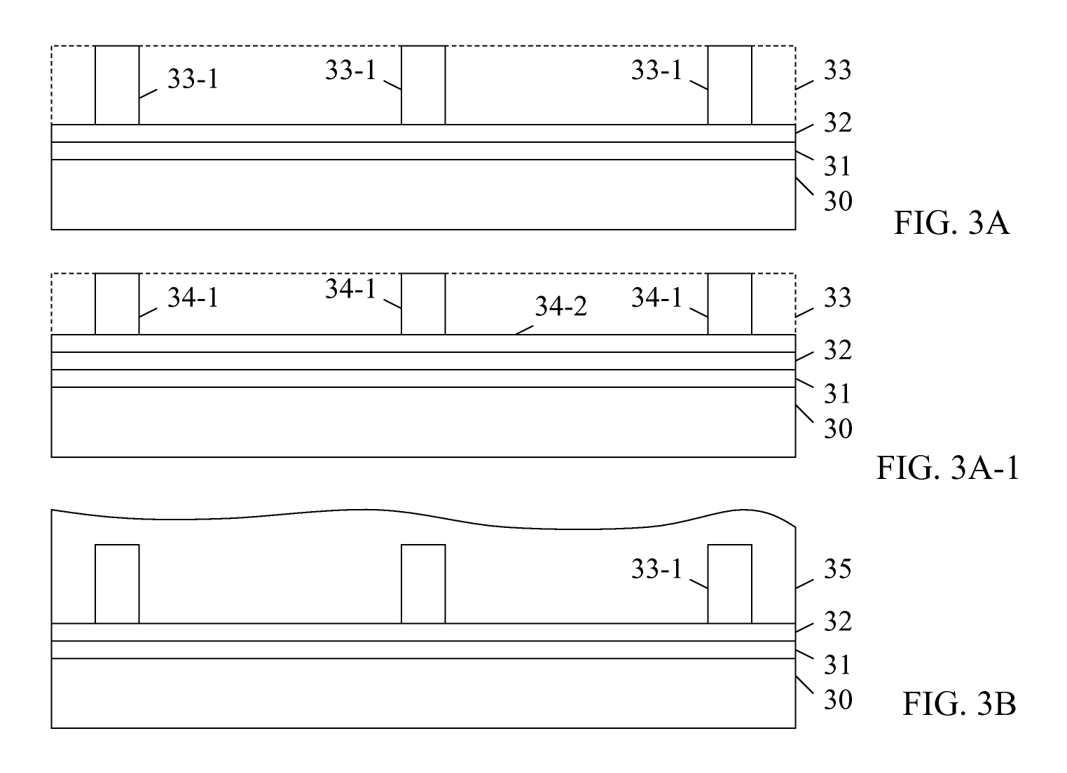

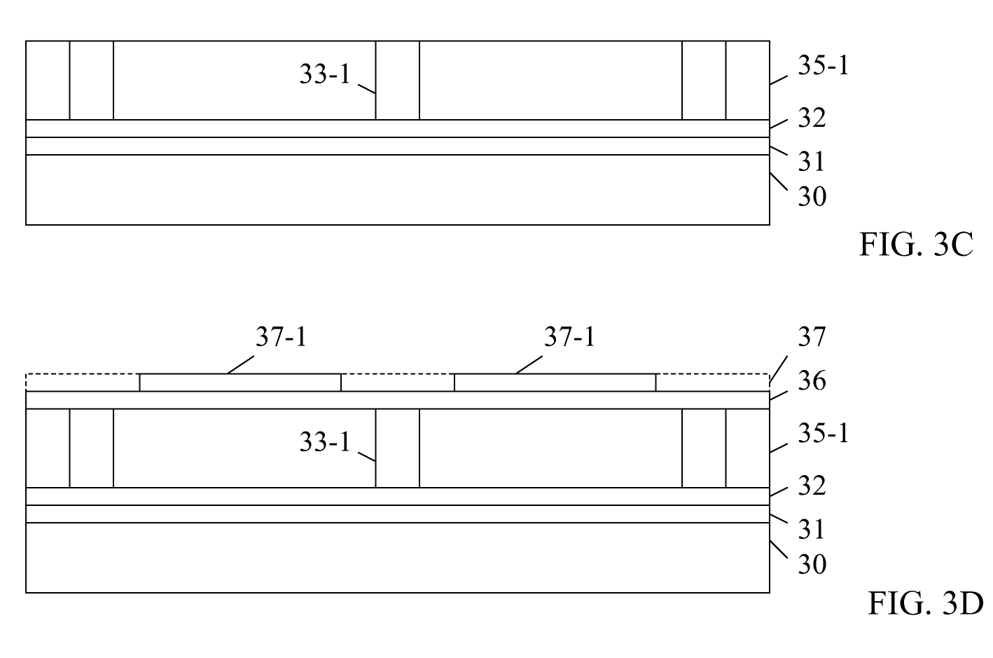

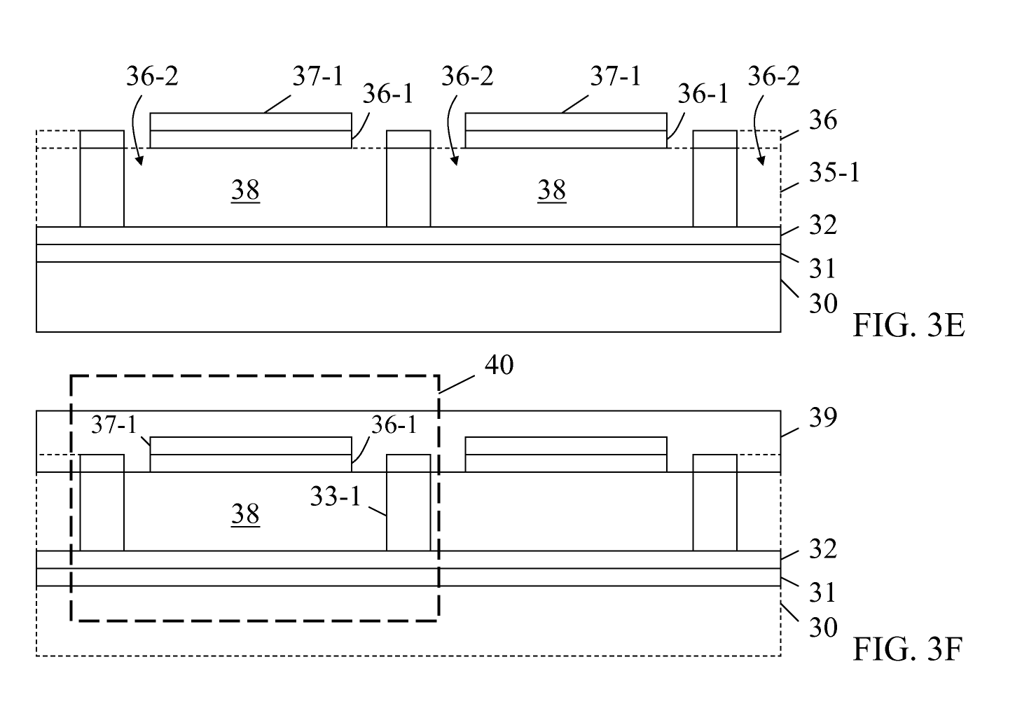

FIGS. 3A to 3F are cross-sectional diagrams illustrating a method of manufacturing an ultrasonic transducer consistent with an example of the present invention. Referring to FIG. 3A, a substrate 30 may be provided to serve as a supporting base on which an ultrasonic transducer array may be fabricated, and may be removed after the ultrasonic transducer array is fabricated. The substrate 30 may include a silicon substrate having a thickness ranging from approximately 200 to 600 micrometer (μm). In one example, the thickness of the substrate 30 may be approximately 525 μm.

A first flexible layer 31, which may eventually serve as a flexible base of the ultrasonic transducer array, may be formed on the substrate 30 by a coating, blading or other suitable processes. The first flexible layer 31 may include a polymeric material selected from one of polyimide, parylene and photoresist such as AZ-4620, polymethylmethacry (PMMA), SU-8, SP-341 and JSR. The first flexible layer 31 may have a thickness ranging from approximately 20 to 400 μm. In one example according to the present invention, the thickness of the first flexible layer 31 may be approximately 90 μm.

Next, a first conductive layer 32 may be formed on the first flexible layer 31 by a sputtering process or other suitable processes. The first conductive layer 32, which may eventually serve as a first electrode for an ultrasonic transducer of the array being fabricated, may include a metal material selected from one of aluminum (Al), aurum (Au), platinum (Pt) and copper (Cu). The first conductive layer 32 may have a thickness ranging from approximately 0.1 to 0.6 μm.

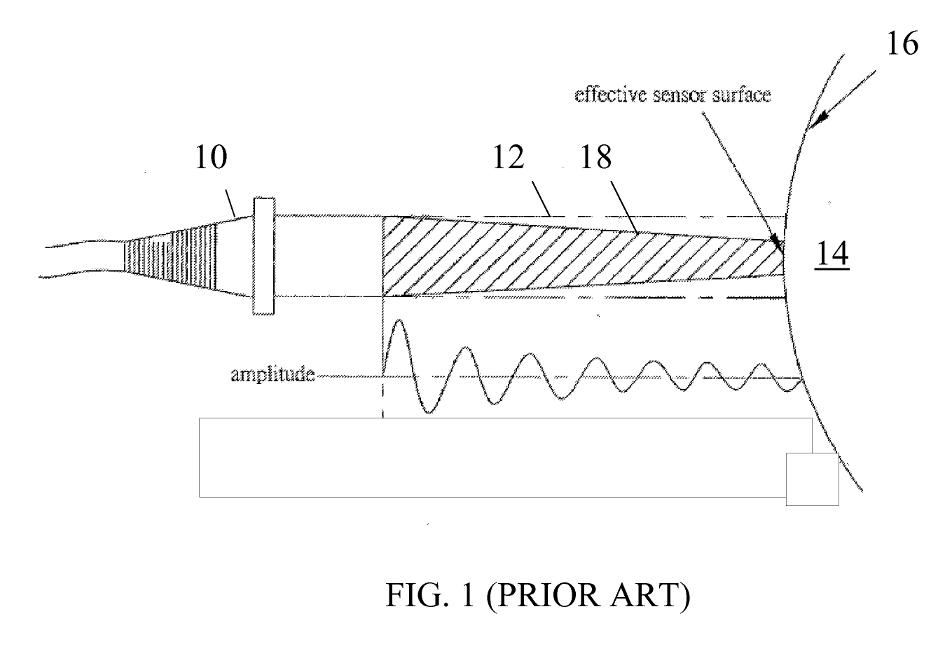

A second flexible layer 33 may be formed on the first conductive layer 32 by a coating process or other suitable processes. The second flexible layer 33 may then be patterned and etched by, for example, a photolithographic process to form a patterned second flexible layer including a number of flexible supports 33-1. In the present example, the patterned second flexible layer may expose portions of the first conductive layer 32. In another example, as illustrated in FIG. 3A-1, the first conductive layer 32 may not be exposed. FIG. 3A-1 is a cross-sectional diagram illustrating a method of manufacturing an ultrasonic transducer consistent with another example of the present invention. Referring to FIG. 3A-1, the method may be similar to that described and illustrated with reference to FIG. 3A except that, for example, a patterned second flexible layer may include a flexible base 34-2 on the first conductive layer 32 and a number of flexible supports 34-1 on the flexible base 34-2. The patterned second flexible layer illustrated in FIGS. 3A and 3A-1 may include a polymeric material selected from one of polyimide, parylene and photoresist such as AZ-4620, polymethylmethacry (PMMA), SU-8, SP-341 and JSR. The thickness of the patterned second flexible layer, or the height of the flexible supports 33-1 and 34-1, may range from approximately 0.5 to 5 μm.

Referring to FIG. 3B, a second conductive layer 35 may be formed over the patterned second flexible layer by a sputtering process or other suitable processes. The second conductive layer 35 may serve as a sacrificial layer and may be removed in a subsequent process. In one example according to the present invention, the second conductive layer 35 may include copper (Cu).

Referring to FIG. 3C, the second conductive layer 35 may be planarized by, for example, a lapping or chemical-mechanical polishing (CMP) process or other suitable processes to form a sacrificial conductive layer 35-1. The sacrificial conductive layer 35-1 may be substantially flush with the flexible supports 33-1 or 34-1.

Referring to FIG. 3D, a third flexible layer 36 may be formed on the sacrificial conductive layer 35-1 by a coating process or other suitable processes. The third flexible layer 36 may include a polymeric material selected from one of polyimide, parylene and photoresist such as AZ-4620, polymethylmethacry (PMMA), SU-8, SP-341 and JSR. The thickness of the third flexible layer 36 may range from approximately 0.5 to 2 μm.

Next, a third conductive layer 37 may be formed on the third flexible layer 36 by a sputtering process or other suitable processes. The third conductive layer 37 may then be patterned and etched to form a patterned third conductive layer including a number of conductors 37-1. Each of the conductors 37-1, which may eventually serve as a second electrode for an ultrasonic transducer of the array being fabricated, may include a metal material selected from one of aluminum (Al), aurum (Au), platinum (Pt) and copper (Cu). The thickness of the patterned third conductive layer may range from approximately 0.1 to 0.6 μm.

Referring to FIG. 3E, the third flexible layer 36 may be etched to form a number of flexible units 36-1, exposing portions of the sacrificial conductive layer 35-1 through openings 36-2. The sacrificial conductive layer 35-1 may then be removed through the openings 36-2 by an etching process, resulting in a number of chambers 38 each of which corresponds to one of the number of flexible units 36-1 and in turn one of the number of conductors 37-1.

Referring to FIG. 3F, a fourth flexible layer 39 may be formed over the patterned third conductive layer by a coating process or other suitable processes. Subsequently, the substrate 30 may be stripped. The fourth flexible layer 39 may include a polymeric material selected from one of polyimide, parylene and photoresist such as AZ-4620, polymethylmethacry (PMMA), SU-8, SP-341 and JSR. The thickness of the fourth flexible layer 39 may range from approximately 2 to 6 μm. In the present example, each of the chambers 38 may be defined by one set of the supports 33-1, one of the flexible units 36-1 and the first conductive layer 32. In another example, also referring to FIG. 3A-1, each of the chambers 38 may be defined by one set of the supports 34-1, one of the flexible units 36-1 and the flexible base 34-2.

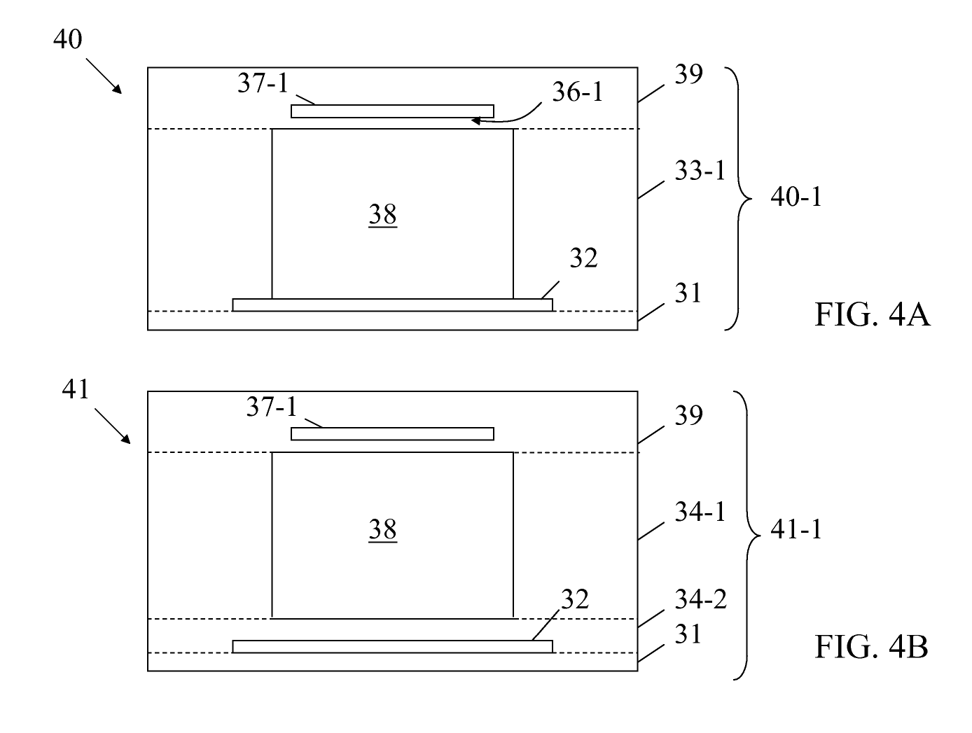

FIG. 4A is a cross-section view of an ultrasonic transducer 40 consistent with an example of the present invention. Referring to FIG. 4A, also referring to a dashed box illustrated in FIG. 3F, the ultrasonic transducer 40 may include the first electrode 32 embedded in a flexible membrane 40-1, the second electrode 37-1 embedded in the flexible membrane 40-1, and the chamber 38 between the first electrode 32 and the second electrode 37-1 in the flexible membrane 40-1. The flexible membrane 40-1 may comprise the first flexible layer 31, the supports 33-1, the flexible unit 36-1 and the fourth flexible layer 39, which are made of substantially the same polymeric material. In the ultrasonic transducer 40, the first electrode 32 is exposed to the chamber 38. Furthermore, when a suitable alternating current (AC) voltage or direct current (DC) voltage is applied across the first electrode 32 and the second electrode 37-1, an electrostatic force may cause the fourth flexible layer 39, or specifically the third flexible unit 36-1 between the chamber 38 and the second electrode 37-1, to oscillate and generate acoustic waves. The third flexible unit 36-1, which has been described and illustrated with reference to FIGS. 3E and 3F, is integrated into the flexible membrane 40-1 together with the first flexible layer 31, the supports 33-1 and the fourth flexible layer 39.

FIG. 4B is a cross-section view of an ultrasonic transducer 41 consistent with another example of the present invention. Referring to FIG. 4B, also referring to FIG. 3A-1, the ultrasonic transducer 41 may include the first electrode 32 embedded in a flexible membrane 41-1, the second electrode 37-1 embedded in the flexible membrane 41-1, and the chamber 38 embedded in the flexible membrane 41-1 between the first electrode 32 and the second electrode 37-1. The flexible membrane 41-1 may comprise the first flexible layer 31, the supports 34-1, the flexible base 34-2, the flexible unit 36-1 and the fourth flexible layer 39, which are made of substantially the same polymeric material.

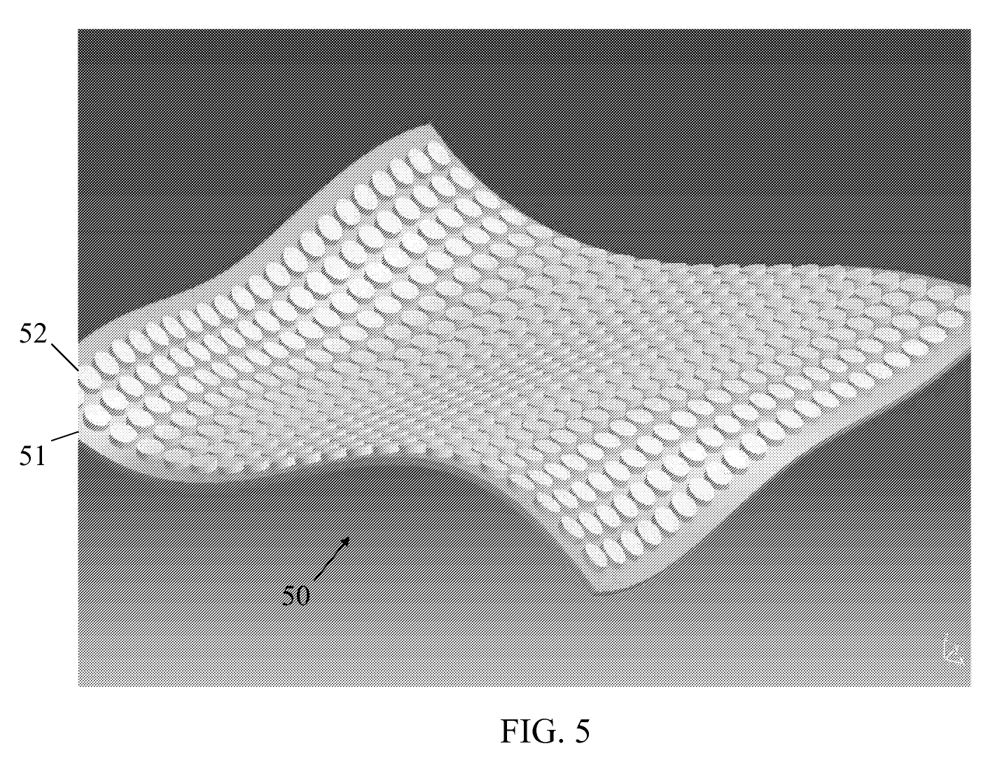

FIG. 5 is a three-dimensional view of an ultrasonic transducer array 50 consistent with an example of the present invention. The ultrasonic transducer array 50 may include a flexible substrate 51 and a number of ultrasonic transducers 52 formed on the flexible substrate 51. In one example according to the present invention, each of the ultrasonic transducers 52 may be similar to the ultrasonic transducers 40 and 41 described and illustrated with reference to FIGS. 4A and 4B, respectively. In another example, each of the ultrasonic transducers 52 may include a number of transducer units each being similar to the ultrasonic transducers 40 and 41 described and illustrated with reference to FIGS. 4A and 4B, respectively. Furthermore, the flexible substrate 51, which is common to all of the ultrasonic transducers 52, may be similar to the first flexible layer 31.

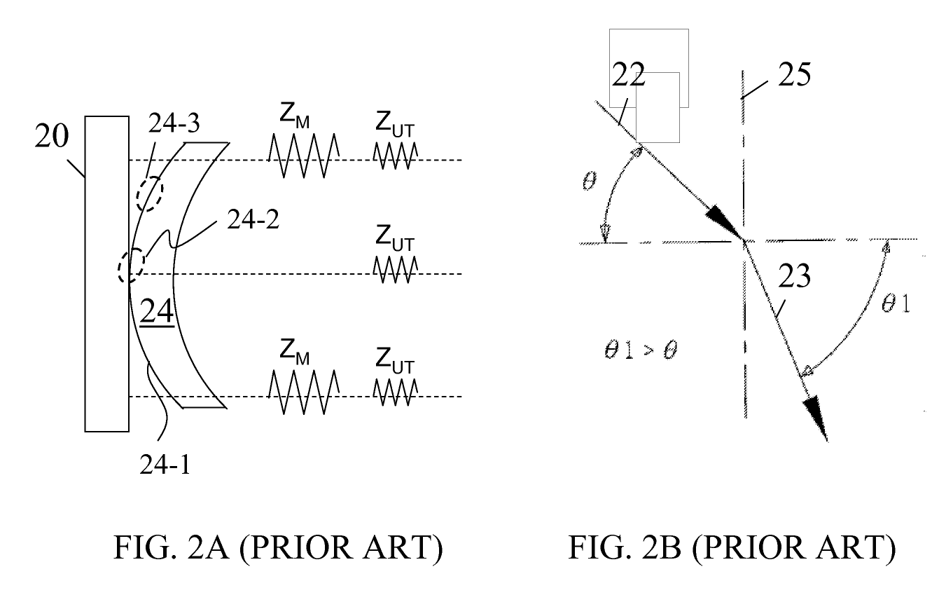

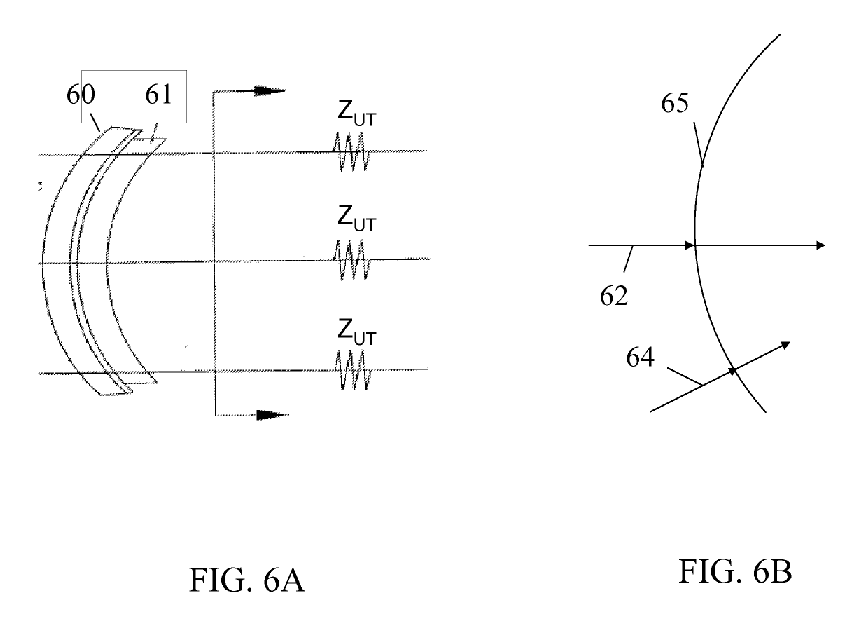

FIG. 6A is a schematic diagram illustrating the operation of an ultrasonic transducer 60 consistent with an example of the present invention. Referring to FIG. 6A, the ultrasonic transducer 60 may be able to conform to a curved surface of an object 61. Compared to the ultrasonic transducer 20 described and illustrated with reference to FIG. 2A, attenuation of an ultrasonic wave transmitted from the ultrasonic transducer 60 may be alleviated.

FIG. 6B is a diagram illustrating ultrasonic waves 62 and 64 from an array of the ultrasonic transducers 60 illustrated in FIG. 6A. Referring to FIG. 6B, the ultrasonic waves 62 and 64 from different ultrasonic transducers 60 of an ultrasonic transducer array may each transmit in the normal direction to the curved surface of the object 61 because the ultrasonic transducers 60 conform to the curved surface. Accordingly, the angle of incidence “θ” and the angle of refraction “θ₁” may substantially equal zero. Compared to the ultrasonic transducer 20 described and illustrated with reference to FIG. 2A, the issue of impedance mismatch may be alleviated.

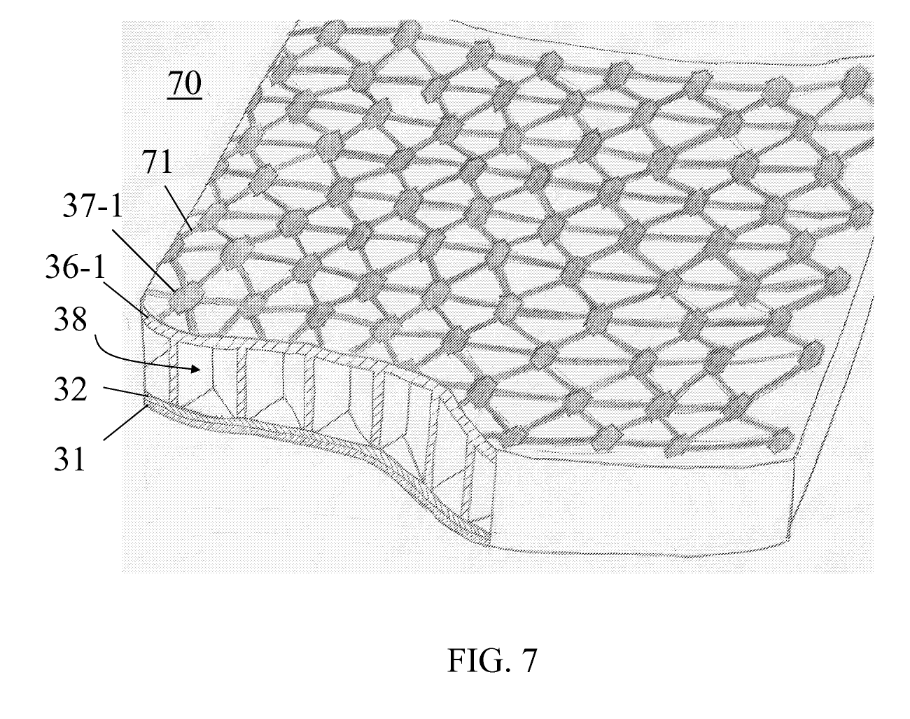

FIG. 7 is a perspective view of an ultrasonic transducer array 70 consistent with an example of the present invention. Referring to FIG. 7, the ultrasonic transducer array 70 in the form of a flexible membrane may include a number of ultrasonic transducers (not numbered) and the first flexible layer 31 and first conductive layer 32 common to the number of ultrasonic transducers. Each of the number of ultrasonic transducers may include one chamber 38 and one conductor 37-1 disposed above the chamber 38. A plurality of conductive wires 71 may be formed simultaneously with the conductors 37-1 to electrically couple the conductors 37-1. Each of the conductive wires 71 may have a width ranging from approximately 3 to 20 μm.

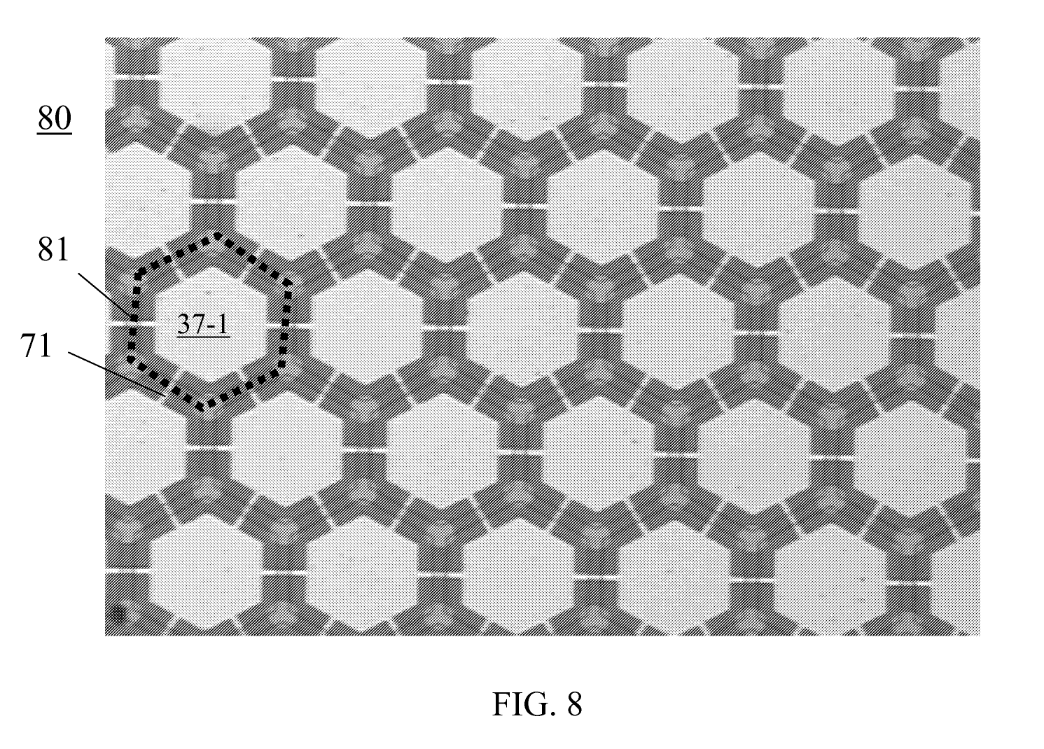

FIG. 8 is a top planar view of an ultrasonic transducer array 80 consistent with an example of the present invention. Referring to FIG. 8, the ultrasonic transducer array 80 may include a number of ultrasonic transducers 81 electrically coupled to one another by the conductive wires 71. For each of the ultrasonic transducers 81, the shape of the second electrode 37-1 may include but is not limited to one of a hexagon, rectangle, triangle, polygon and circle. Furthermore, the shape of the chamber (not shown) may also include but is not limited to one of a hexagon, rectangle, triangle, polygon and circle.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

We claim:

1. An ultrasonic transducer comprising:
a flexible membrane;
a first electrode in the flexible membrane;
a second electrode in the flexible membrane; and
a chamber in the flexible membrane between the first electrode and the second electrode.

2. The ultrasonic transducer of claim 1, wherein the flexible membrane includes a polymeric material selected from one of polyimide, parylene and photoresist.

3. The ultrasonic transducer of claim 1, wherein the flexible membrane is conformable to a surface topology of an object.

4. The ultrasonic transducer of claim 1, wherein at least one of the first electrode or the second electrode includes a conductive material selected from one of aluminum (Al), aurum (Au), platinum (Pt) and copper (Cu).

5. The ultrasonic transducer of claim 1, wherein the flexible membrane includes multiple flexible layers and the chamber is surrounded by the multiple flexible layers of the flexible membrane.

6. The ultrasonic transducer of claim 1, wherein the first electrode is exposed to the chamber.

7. An ultrasonic transducer comprising:
a first flexible layer;
a first conductive layer over the first flexible layer;
a second flexible layer over the first conductive layer;
a chamber in the second flexible layer;
a third flexible layer over the chamber; and
a second conductive layer in the third flexible layer,
wherein a portion of the third flexible layer between the second conductive layer and the chamber is capable of generating acoustic waves in response to a voltage applied across the first conductive layer and the second conductive layer.

8. The ultrasonic transducer of claim 7, wherein each of the first, second and third flexible layers includes a polymeric material selected from one of polyimide, parylene and photoresist.

9. The ultrasonic transducer of claim 7, wherein at least one of the first conductive layer or the second conductive layer includes a material selected from one of aluminum (Al), aurum (Au), platinum (Pt) and copper (Cu).

10. The ultrasonic transducer of claim 7, wherein the first conductive layer is exposed to the chamber.

11. An ultrasonic transducer array comprising:
a flexible substrate;
a first conductive layer on the flexible substrate; and
a number of ultrasonic transducers on the first conductive layer, each of the number of ultrasonic transducers comprising:
a flexible membrane over the first conductive layer;
a second conductive layer in the flexible membrane; and
a chamber in the membrane between the first conductive layer and the second conductive layer.

12. The ultrasonic transducer array of claim 11, wherein at least one of the flexible substrate or the flexible membrane includes a polymeric material selected from one of polyimide, parylene and photoresist.

13. The ultrasonic transducer array of claim 11, wherein the flexible substrate is conformable to a surface topology of an object.

14. The ultrasonic transducer array of claim 11, wherein at least one of the first conductive layer or the second conductive layer includes a conductive material selected from one of aluminum (Al), aurum (Au), platinum (Pt) and copper (Cu).

15. The ultrasonic transducer array of claim 11, wherein the first conductive layer is exposed to the chamber of each of the number of ultrasonic transducers.

16. The ultrasonic transducer array of claim 11 further comprising a plurality of conductive wires electrically coupling the second conductive layer of each of the number of ultrasonic transducers.

17. An ultrasonic transducer array comprising:
a first flexible layer;
a first conductive layer on the first flexible layer; and
a number of ultrasonic transducers on the first conductive layer, each of the number of ultrasonic transducers comprising:
a second flexible layer over the first conductive layer;
a chamber in the second flexible layer;
a third flexible layer over the chamber; and
a second conductive layer in the third flexible layer,
wherein a portion of the third flexible layer between the second conductive layer and the chamber is capable of generating acoustic waves in response to a voltage applied across the first conductive layer and the second conductive layer.

18. The ultrasonic transducer array of claim 17, wherein each of the first, second and third flexible layers includes a polymeric material selected from one of polyimide, parylene and photoresist.

19. The ultrasonic transducer array of claim 17, wherein at least one of the first conductive layer or the second conductive layer includes a conductive material selected from one of aluminum (Al), aurum (Au), platinum (Pt) and copper (Cu).

20. The ultrasonic transducer array of claim 17, wherein the first conductive layer is exposed to the chamber.
